(19) United States
(12) Patent Application Publication
(10) Pub. No.: US 2004/0033482 A1
(43) Pub. Date: Feb. 19, 2004

(54) Device and method for the measurement of forces from living materials

(76) Inventor: Gerhard Artmann, Vaals (NL)

Correspondence Address: Friedrich Kueffner, Suite 910, 317 Madison Avenue, New York, NY, 10017, US

(21) Appl. No.: 10/362,949

(22) PCT Filed: Jul. 6, 2001
(86) PCT No.: PCT/EP01/07762

(30) Foreign Application Priority Data
Aug. 26, 2000 (DE) 100 41 988.7

(51) Int. Cl. C12M 1/34; C12Q 1/02

(52) U.S. Cl. 435/4; 73/862.637; 435/287.1

(57) ABSTRACT

The invention relates to a device and method for the measurement of lateral intrinsic forces from living materials, in particular in cell layers or tissue structures.

The present invention relates to a device and to a method for measuring lateral intrinsic forces of living material.

Cell forces are mechanical forces which emanate from cells and are transmitted to other cells (intercellularly) or substrates. The forces are generated within cells (intracellularly) and, outside the cells, transmitted to neighboring cells or transmitted, by way of the extracellular matrix (ECM) which is secreted by the cells, to a substrate. Such substrates can be natural or artificial surfaces, such as bones, noncellular connective tissue structures, artificial implants or biomaterials. The biological elements of the origin and conduction of the forces are the proteins. In addition to the internal osmotic pressure in the cytosol, it is the crosslinked structural proteins (cytoskeleton) which give rise to forces in the cells and pass these forces on through the cell. These forces are passed on, by way of integral membrane proteins which are coupled mechanically to the extracellular space, to neighboring cells or other substrates.

The cells, and the forces which emanate from them, can be stimulated by a very wide variety of stimulating agents. This results in the structural proteins being altered, and the cell forces are augmented (induction) or diminished (relaxation). This can lead to contractions, to a movement in the direction of a physical or chemical stimulus, or to other phenomena. The underlying mechanisms are only partially known. Examples of macroscopic effects are muscle contraction, blood vessel contraction or dilatation, passage of cells through tissue or, for example, the adherence of the cells to, or their detachment from, artificial biomaterials. In the case of biomaterials, both phenomena may be desirable depending on the application case.

Cellular forces have thus far been determined on isolated individual cells (J. van Velden et al.: Force production in mechanically isolated cardiac myocytes from human ventricular muscle tissue, Cardiovascular Research 38 (1998) 414-423). In this connection, the cells which were employed, in this case myocytes, were attached with silicone adhesive to thin stainless steel needles (tip diameter ˜15 μm). This can elicit undefinable preparation artefacts which falsify the measurement result as compared with the actual state in the cells. Furthermore, the measurement of the cell force in this case is very elaborate and is effected by using a force transmitter or what is termed a force transducer (SensoNor, Horten, Norway), and a piezoelectric motor (Physik-Instrumente, Waldbrunn, Germany), and also a thin quadratic carbon fiber (length 15 mm, thickness 0.5 mm), in order to achieve adequate sensitivity.

Kolodney, M. S., et al. (1992, Isometric contraction by fibroblasts and endothelial cells in tissue culture: a quantitative study. J. Cell Biol 117:73-82) describe a method in which individual cells grow into three-dimensional protein gels (collagen gel network), with the protein gels being held between two holders. The tensile forces which are produced by the cells in the three-dimensional gels as a result of growth, movement and/or mechanical activity are determined using a sensor. Extensible measuring strips, which generate an electrical signal which can be used for deducing the tensile force, are employed as the force sensor. However, a disadvantage of this method is that only simple traction experiments known from mechanics can be reproduced. The device generates transverse contractions, with the thickness of the protein gel changing along the axis of the traction path. However, the tension distribution in the gel changes at the same time. In this connection, the tension status in three-dimensional structures, like the collagen matrix mentioned here, is to a large extent site-dependent. This means that, while the force measured by the sensor depends on the cell forces per se, it also depends on the cell number, the cell orientation, the period of measurement, since cells can react to tension gradients by migrating, the homogeneity of the cell distribution, the supply of nutrients to the cells through the matrix, and the geometry and largely unknown physical properties of the collagen matrix in which the cells are embedded. This tension, which is local but unknown in a three-dimensional matrix, which an individual cell in the matrix experiences leads to a nonuniform and chronologically varying reaction of the cells, depending on the site at which they are located. This means that, while the varying tensions which act on an individual cell enable the cell forces in the matrix to be measured reproducibly, the force measurements are only average or relative force measurements, assuming the measurement is always carried out using the same measuring device. It is not possible to use the experiments disclosed by Kolodney et al. to draw conclusions with regard to the forces which are present in a coherent biological system (cell layers). In addition to this, the method which is described here is restricted to investigating particular cells since not all cells, without exception, grow in a three-dimensional matrix; some cells, such as endothelial cells or epithelial cells, only grow (superficially) as cellular monolayers.

Consequently, there are no known measurement methods which make it possible to determine cellular forces in coherent cell layers or even cell tissues or whole organs. While Leibner, Th. et al (Abstract in Proceedings of the 4^{th} International Conference on Cellular Engineering, Nara, Japan, 30.11-03.12.1999) indicate that it is possible to measure the forces emanating from a layer of human fibroblast cells, no special apparatus or precise procedure is disclosed in this publication.

Furthermore, it is not possible to use the methods which have thus far been disclosed to quantify cell forces which are occasioned by cell communication, such as a synchronous contraction of several cells or an autocontraction or autorelaxation which is produced by the cells.

The object of the present invention is therefore to make available a device and a method for determining lateral intrinsic forces in coherent cell layers/cell tissues or even (cultured) organs, which device and which method do not suffer from the previously mentioned disadvantages. In addition to this, it would be desirable to have a measurement method which could be used to correlate, in a single experimental setup, i.e. on the same cell layers, the biological events (e.g. changes in the composition of the cell matrix or of the cytoskeleton, or changes in gene expression inter alia), for example due to the culturing conditions, directly with the cell forces.

This object is advantageously achieved by means of the present invention.

The present invention relates to a device for measuring forces of living material, which device contains a mounting, an elastically deformable membrane and a sensor for measuring the change of forces acting on the membrane, comprising a membrane which is arranged on a mounting, which is stretched in a planar manner, which is freely accessible from both sides and which can be deformed elastically by applying a physical force, and which can be bonded to the living material after the latter has been applied to this membrane.

Within the sense of the invention, the previously mentioned forces are to be understood as meaning the linear load (measured in Pa·m). It has the dimension of force per length. This unit follows from the fact that the cell layer is thin as compared with its lateral breadth and can therefore be regarded, to a good approximation, as being an infinitely thin layer.

In a particular embodiment of the present invention, the elastically deformable membrane is drawn, during an adhesion process, over a circular mounting, for example the underside of a cylinder, such that the membrane is completely flat (planar). The initial tension is selected such that the membrane is flat but not plastically deformed. The membrane is also still flat, and not plastically deformed, when additional tensions, for example due to culture medium or living material applied to the membrane, act on the membrane. A diagram of the device is outlined in FIG. 1.

The device according to the invention furthermore comprises a membrane which exhibits a hydrophilic surface and/or a surface which is suitable for adhering and/or culturing living material, and/or is correspondingly treated and/or supplied with an adhesion-mediating substance.

Since the living material is hydrophilic as a result of its content of protein and carbohydrate, the membrane which is employed in the device according to the invention should also be hydrophilic or be correspondingly modified or prepared by means of suitable measures. This can be achieved, for example, by using known methods to plasma-etch the surface for the purpose of producing polar groups, or supplying the membrane surface with adhesion-mediating (adhesive) substances, such as gelatin.

In addition to this, the membrane according to the invention exhibits a number of other properties. For example, it is biocompatible, noncytotoxic, resistant to metabolic products and environmental conditions of the living material (biologically inert), transparent, resistant to heat and pressure (autoclavable), resistant to tearing and resistant to acids, bases or organic solvents, and exhibits low gas permeability. In this connection, the device according to the invention comprises a membrane which contains enzymically degradable material. For example, collagen, elastin, or fibrinogen, and/or combinations thereof, can be introduced into and/or applied onto the membrane. However, this enumeration is not limiting for the present invention.

In addition, the device according to the invention comprises a membrane which is pore-free and/or exhibits a thickness in the range from 0.1 to 10 μm, preferably of from 0.5 to 5 μm and particularly preferably of 1 μm, with the ratio of the thickness of the membrane to the diameter or circumference or edge length of the membrane (depending on the shape of the membrane) having a value in the range from 6×10⁻⁶ to 6×10⁻⁴, preferably from 3×10⁻⁵ to 3×10⁻⁴ and particularly preferably 6×10⁻⁵. In this connection, the membrane of the device according to the invention can have any arbitrary shape, preferably circular, hemispherical, spherical, rectangular or square. In addition, the membrane is mechanically stable and elastically deformable. In addition to this, the device according to the invention is characterized by the fact that, at 25° C., the modulus of elasticity of the membrane has a value in the range from about 1000 to 10 000 MPa, preferably of from about 2500 to 6500, particularly preferably of about 3900 MPa.

In a particularly preferred variant of the present invention, the membrane is a polyethylene film (PET). Any biomaterial, in particular biomaterials which have been tested for use as blood vessel prostheses, is also conceivable. However, these embodiments are not limiting for the present invention.

The previously described device according to the invention is also distinguished by the fact that the living material which is applied to the membrane contains whole cells, one or more cell layer(s) (monolayer or multilayer), secreted cell material, preferably extracellular matrix (ECM), cell constituents and/or matrix constituents, with it being possible to genetically alter the living material. In a particular embodiment of the present invention, the living material comprises fibroblasts and/or muscle cells, preferably smooth muscle cells and/or endothelial cells, simply to mention a nonlimiting selection.

The present invention furthermore relates to a method for measuring forces of living material, with laterally acting, intrinsic forces of the living material being directly transmitted, where appropriate before, during and/or after stimulation of the material with external stimuli, to an elastically deformable membrane and the resulting change in the deflection of the membrane being registered quantitatively. That is, the present method according to the invention makes it possible to achieve a direct correlation (and quantification) between biological events in cell layers (e.g. change in the composition of the matrix or of the cytoskeleton or in gene expression) and the changes in the cell forces which accompany them.

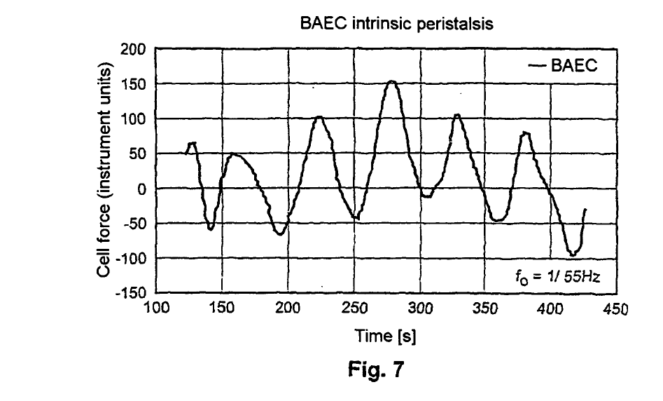

According to the invention, “intrinsic forces” are to be understood as meaning both intracellular and intercellular forces. In this connection, it is also possible, according to the invention, to measure what are termed rhythmic intrinsic contractions and/or relaxations of cells or cell layers. Thus, endothelial cells in a monolayer formation, for example, exhibit rhythmic intrinsic contractions and relaxations without the cells being stimulated in any way. FIG. 7 shows a graph of the intrinsic peristalsis of bovine aorta endothelial cells as measured using the method according to the invention.

In addition, the method according to the invention is characterized by the fact that an elastically deformable membrane is stretched in a planar manner on a mounting such that it is freely accessible from both sides, the membrane is subsequently elastically deformed by applying a physical force, living material is applied to the elastically deformed membrane and a bond is formed between the living material and the membrane, for example by culturing the living material and/or further adhesion mediation in and/or on the membrane. In addition to this, the living material is, where appropriate, subjected, during and/or after the formation of the bond with the membrane, to external forces which are applied constantly and/or in a pulsating manner and/or in an oscillating manner; in addition, the lateral, intrinsic forces emanating from the living material are transmitted to the membrane, where appropriate additional external stimuli are exerted on the living material, and the forces and/or force changes of the living material which have been transmitted to the membrane, and the time constants and/or relaxation times which are associated therewith, are quantitatively determined continuously, as changes in the deflection of the membrane, using a sensor.

In the course of the method according to the invention, the membrane is bonded to the mounting, while it is being stretched in a planar manner and/or after it has been stretched in a planar manner, and subsequently elastically deformed by being overlayed with a liquid column, resulting in the membrane being deflected. The stretching of the membrane in this connection can be neglected. The hydrostatic pressure which is applied, according to the invention, to the membrane preferably corresponds, at a modulus of elasticity of about 3900 MPa, to a liquid column having a fill height in the range from about 0.1 to 50 mm, preferably of from about 0.5 to 10, and particularly preferably of about 2 mm. For example, at a modulus of elasticity of about 1000 MPa, the liquid column corresponds to a fill height of from about 0.1 to 10 mm and, at a modulus of elasticity of about 10 000 MPa, to a liquid column having a fill height of from about 1 to 150 mm. In general, the fill heights which are to be applied change in accordance with the known laws of mechanics.

The previously mentioned liquid column is, for example, a medium which is suitable for culturing the living material. Immediately after the membrane has been deformed by applying a physical force, by overlaying with (culture) liquid, the living material is then cultured in the culture medium and on the membrane. After that, the change in the deflection of the membrane due to the cell force which is operative is then measured. The tension in the cell layer is then calculated from the change in height of the membrane deflection. In this connection, the deflection of the membrane due to the cell force decreases when the cells contract; i.e. the fill height of the liquid column or of the culture medium is slightly raised. The converse happens when the cells (and/or the extracellular matrix) relax(es). This change in the membrane deflection is then, according to the invention, recorded quantitatively, and preferably continuously, using a sensor.

A variant of the present invention comprises a method for measuring forces of living material, with the forces transmitted to the membrane by the living material being measured as a change in the fill height of the liquid column located above the membrane. In this connection, the deflection of the membrane is used as a controlled variable. The change (raising or lowering of the fill height), which is brought about by the forces of the living material, in the liquid column located above the membrane is offset by adding or withdrawing a corresponding quantity of liquid such that the deflection of the membrane retains the same value which it had before the forces of the living material were transmitted to the membrane. This means that no change in the deflection of the membrane can be measured and/or the deflection of the membrane is, or is kept, constant.

The force of the living material which is transmitted to the membrane can be determined by precisely determining the quantity of the liquid which is added or withdrawn. The appropriate formulae and conversion factors for this purpose are known to the skilled person and are not cited any further. This procedure is suitable for determining the forces of living material which are due both to contraction of the living material and to relaxation of the living material.

In this connection, the invention also encompasses a suitable device for measuring the change in the fill height of the quantity of liquid which is located above the membrane. Where appropriate, this device contains, in contrast to the device which has already previously been described, suitable components for automatically removing or adding quantities of liquid, which components communicate, where appropriate, with the sensor thereby ensuring regulation for retaining the deflection of the membrane which was initially set.

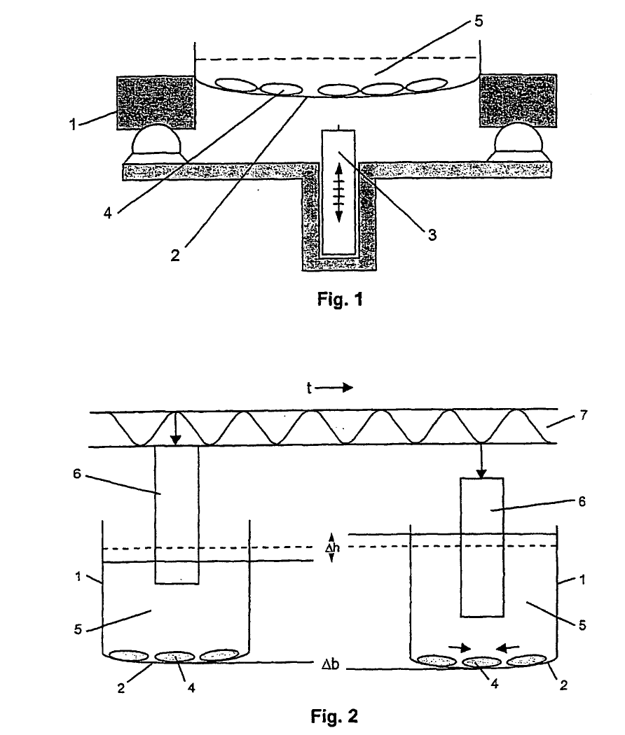

In a particular embodiment of the method according to the invention, the living material is subjected to external mechanical, electrical and/or magnetic forces, and a cell force response is thereby stimulated. The method is preferably characterized by the fact that the living material is subjected to changes in external hydrostatic pressure by a plunger being immersed, cyclically and with varying amplitude and frequency, in the liquid column over the membrane. A diagram of the procedure is shown in FIG. 2. Immersing the plunger more deeply in the solution above the membrane results in a higher hydrostatic pressure, while more shallow immersion results in a lower pressure. However, the changes in pressure can also be generated by applying a negative pressure to the membrane of the device according to the invention. Alternatively, instead of a mechanical plunger, a magnetic or electrical field can be directed toward the living material. The external forces can be applied either during the culturing of the cells, for example in an incubator, or in the measuring instrument itself, after the cells have been cultured. In the device according to the invention, the mounting, together with the membrane and the living material, can be separated from the sensor component of the device according to the invention such that the culturing and, where appropriate, cell training, take place in the incubator and the mounting which has been prepared in this way is then inserted into the device in order to carry out the measurement using the previously mentioned sensor.

The living material is mechanically stressed by the very small extensions and compressions which arise and reacts with cell and matrix growth and/or gene expression which is/are altered as compared with an unstressed state. As a result of the method according to the invention, the living material experiences a “training effect”, as it were. An important feature of the present invention is that the effects of this “training” can be directly determined quantitatively by measuring the cell forces. This means that the cells are initially trained and the cell forces of the trained cells are measured directly following that, i.e. in one and the same experimental setup. This represents a crucial advantage as compared with the previously known methods for cell training. Thus, it has previously only be possible to use the known cell extension devices for determining effects such as gene expression, calcium ion flow via the cell membranes or protein secretion (Tschumperlin, D. J. et al., Deformation-Induced Injury of alveolar epithet cells, effect of frequency, duration and amplitude, Am. J. Respir. Crit Care Med, Aug. 1, 2000, 162(2): 357-362 and Hipper, A. et al., Cyclic mechanical strain decreases in the DNA synthesis of vascular smooth muscle cells, Pflugers Arch., May 2000, 44(1): 19-27) but not the consequences of cellular force development, as is now possible by means of the present invention.

In another variant, the method according to the invention is characterized by the fact that the living material is subjected to the external addition of chemical and/or biochemical and/or biological compounds, preferably in the form of an aqueous solution. In this connection, the hydrostatic pressure above the membrane is kept constant, during the addition of aqueous solutions containing chemical and/or biochemical and/or biological compounds [lacuna] a corresponding quantity of aqueous solution which is already present above the membrane being simultaneously withdrawn.

The chemical and/or biochemical compounds which are preferably contained in the aqueous solution as “chemical stimulants” are, for example, thrombin, trypsin, EDTA or collagenase and/or combinations thereof. Furthermore, pharmacologically active compounds, such as inositol triphosphate P3, nocadazole/taxol, cytocalasin D, calcium/calmodulin and fibronectin/cycloheximide, may also be mentioned as being chemical stimulants. In addition, the cells can be stimulated with nitrogen monooxide (NO) or a change in oxygen partial pressure. A “biological compound” is to be understood, for example, as being genetic material which can be used to achieve changes in gene expression. Furthermore, it is also possible to conceive of stimulating the cell layers, in particular the protein moieties (e.g. the intercellular and/or cell matrix-adhesion proteins and/or the cytoskeletal proteins) by means of interactions with specific antibodies. In this way it is possible, inter alia, to analyze the contribution of individual proteins to cellular force development and/or force conduction. In the same way, the cell layers which are growing on the membrane can have been and/or can be genetically manipulated. It is then possible, by means of making specific changes to the culture medium, to stimulate gene expression, for example, such that the influence of individual gene activities on the cell forces can be investigated in a specific manner. This is of great interest in the development of drugs, in particular. The above enumerations of stimulants only serve to explain the present invention and do not have any limiting effect on it.

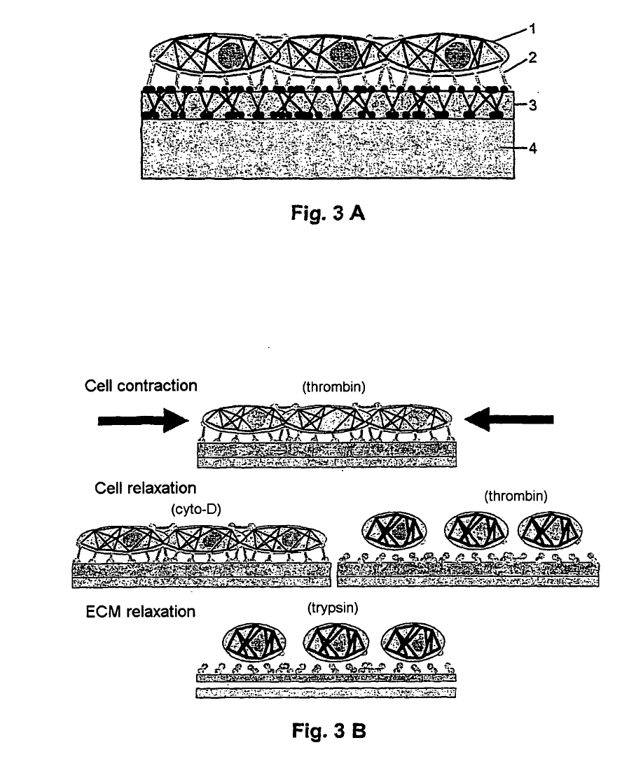

The effect of external chemical stimuli on the intrinsic lateral forces of the living material is depicted diagrammatically in FIG. 3 taking as examples thrombin, EDTA and trypsin. In this connection, thrombin brings about a contraction, which is indicated by the arrows, of the living material, while cytochalasin D and EDTA bring about a relaxation of cell tension due to destruction of the cytoskeleton of the cells and due to the detachment of the cells from the extracellular matrix (ECM), respectively. Finally, trypsin brings about a relaxation of the extracellular matrix as a result of a partial degradation of the extracellular matrix and detachment from the membrane.

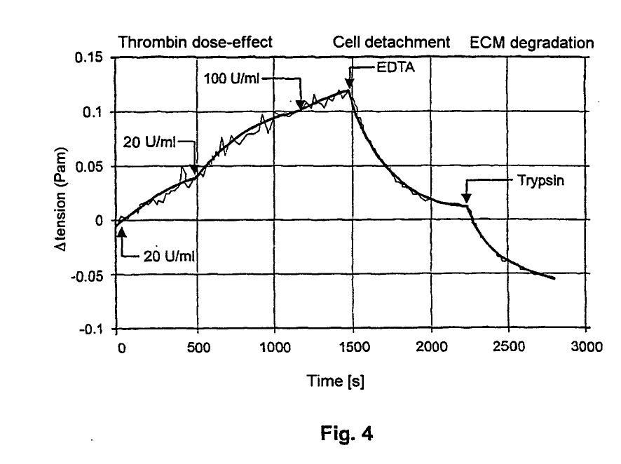

FIG. 4 shows a change in cell layer tension in dependence on time under the action of thrombin, EDTA and trypsin. In this case, the contraction of the living material can be observed to increase as the concentration of thrombin increases, as the exponential increase in cell tension demonstrates. If the bonds between the cells and the extracellular matrix are broken with EDTA, forces can no longer be passed on to the membrane and the intrinsic forces which are measured decrease. The remaining residual force, which emanates from the extracellular matrix, also declines as soon as this layer is further degraded by adding trypsin.

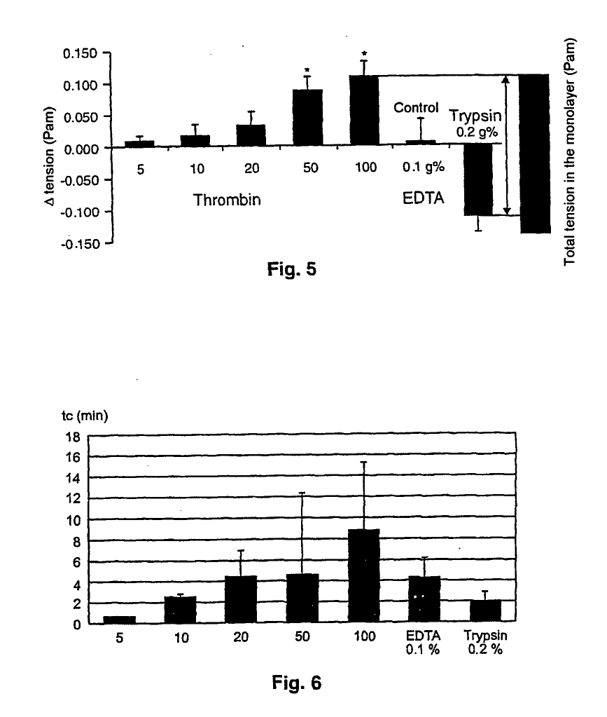

This demonstrates clearly that the method according to the invention can be used not only to measure the absolute changes in the intrinsic forces, or those changes which are achieved after stimulation has taken place, but also to measure conduction times and retardation times. For physiological reasons, measurement of the lateral intrinsic forces of the living material, in particular after chemical or mechanical stimulation have taken place, is a time-dependent process whose course can be measured continuously in accordance with the invention. Conclusions with regard to the cell force itself, with regard to the rate of force generation or with regard to force relaxation after the external stimulus has been removed, can be drawn from the results obtained in accordance to the invention. This is depicted once again in FIG. 5 and FIG. 6. Thrombin causes the tension transmitted to the membrane to increase markedly. Adding EDTA results in the membrane relaxing, due to the detachment of the cells from the extracellular matrix. The addition of trypsin causes the tension in the extracellular matrix to subside as a result of the matrix becoming degraded.

Consequently, the present invention is suitable for measuring lateral forces of both the cells and their extracellular matrix (ECM), which is originally secreted by the cells. The present invention also makes it possible to measure the lateral forces on the membrane after the cells have been detached from the ECM, such that, in this way, the forces of the ECM can be measured on their own. In addition to this, the extracellular matrix can also be degraded, for example enzymically, and the remaining tensions in the membrane can be measured on their own. In this way, it is possible to specifically determine the forces which arise at the beginning of the measurements, as a result of the membrane being overlayed with an aqueous solution, even before living material has been applied to the membrane (the initial membrane tension). These values are to be regarded as being calibration values of the prestressed membrane before beginning measurement of the lateral forces of the living material. Consequently, the membrane contained in the device according to the invention is precisely characterized mechanically.

A very particular advantage of the present invention is that it simultaneously combines in itself all the following properties.

The device according to the invention and the method according to the invention make it possible to investigate cells in a cell formation, with it being possible to directly visualize and count the cell number. It is also possible to stain the cytoskeleton and in this way make it visible. It is possible to discern the geometry and possible artefacts of the manner in which the cells overgrow the membrane. During growth, the cells form an extracellular matrix which, after the cells have been detached, can be separately investigated physically, chemically and/or mechanically. The thickness of the cell layer (z direction) is very slight (only a few μm) and, in the method according to the invention, is very small as compared with the length (in the x and y directions; approx. 10 mm). It follows from this that the tension state, which is to be measured, of the cell layer can be approximately regarded as being two-dimensional. The tension states in the membrane itself can be defined precisely (calibration). In addition, the present invention combines the following advantages, such as resolution of the force measurement in the nanonewton or piconewton range, time resolution in the millisecond range, a high degree of measurement accuracy with a margin of error of less than 10%, measurement of absolute and (chemically and/or physically) stimulated forces of the living material in one setup, investigation of high cell densities, involving a cell number in the region of more than 1000 cells, and also simplicity of preparation, which is suitable for routine purposes, combined with low cost.

According to the invention, the present method is furthermore characterized by the fact that the time course of the forces which are transmitted to the membrane can be measured, preferably continuously. This can take place over a period lasting from 1 second up to several hours and depends, in particular, on the lifetime of the cells. The sampling rate (defined as the number of measuring points per time) can be 100/second and is unlimited in the direction of smaller sampling rates (lower limit). In this connection, an appropriate lower limit for the sampling rate is 1/minute.

In this connection, it is to be noted that the osmotic pressure of the liquid above the membrane is kept constant even over a long period of measurement by adding a quantity of liquid which corresponds to that which has been lost, for example, by evaporation. In another variant of the present invention, the intrinsic forces and/or force changes (linear load and/or linear load changes) emanating from the living material are measured in a range from 0 to 5000 mPa·m, preferably of from 0 to 500 mPa·m. Furthermore, the forces and/or force changes are measured by sampling the membrane in a manner which does not involve any contact. Examples of contactless and reaction-free measuring methods are sampling the membrane deformation by means of laser beams, interference technology, atomic force microscopy or scanning-tunnel microscopy. This emuneration serves for explanation and does not constitute any limitation to the present invention.

The present invention furthermore relates to a method for identifying compounds (what is termed a “screening method”) which exert a measurable effect on the intrinsic forces of living material, with the compounds being added, in a method according to the invention of the previously described nature, to the living material and it then being possible to determine the extent of the change in the intrinsic forces directly. Thus, it can be directly measured whether, and to what extent, for example in dependence on the concentration employed, the compounds which are used exert an effect on the cell forces.

The present invention furthermore relates to the use of the device according to the invention, possessing the previously described properties, for measuring lateral intrinsic forces in living material, in particular cell layers and/or organs/organ parts. This also includes those which are cultured.

The present invention furthermore encompasses the use of the device according to the invention for identifying chemical and/or biochemical and/or biological compounds, for example genetic material and/or specific antibodies, which exert an influence on the lateral intrinsic forces of living material, in particular cell layers and/or organs/organ parts.

The use of the compounds which have been identified in accordance with the invention is of particularly great interest for producing compositions for employment in areas of pharmacology or toxicology and/or transplantation medicine.

The present invention is characterized in more detail by means of the following implementation examples, which are not, however, limiting:

Preparing the Measuring Device and Applying Living Material:

A commercially available PET membrane is adhered to an open end of a cylinder; the other end remains open. During the adhesion process, a metal ring is used to pull uniformly on the membrane, such that the membrane is then stretched so that it is completely flat but not plastically deformed. 400 μl of cell culture medium (DPBS), corresponding to a fill height of a liquid column of about 2 mm, are then applied to the membrane (having a diameter of 16 mm). Due to the culture medium, the membrane is elastically preformed, i.e. deflected, and prepared for the application of living material.

1 ml of a suspension of fibroblasts (or smooth muscle cells), containing a cell count of 5×10⁴/ml (or 7×10⁴/ml), is then added to the prestretched membrane and the entire device is incubated in an incubator at 37° C. for 4 days for the purpose of culturing the cells. After the culturing, the culture medium is replaced with 400 μl of fresh medium. The mounting, together with membrane and cells cultured on it, which has thus been prepared is inserted into the measuring device and the measurement is carried out using a laser beam as the sensor.

As a result of replication, the cells adhere to the membrane and form a confluent monolayer (or multilayer). In connection with this, the cells secrete, over time, an extracellular matrix (ECM).

As a result of the growth of the cells, and the tensile stress in the cell layer and in the ECM which is connected therewith, and as a result of the fact that the cells and the ECM adhere to the membrane, the membrane experiences a change in tension. As a consequence of this, the deflection of the membrane decreases and raises the fill level of the medium slightly. This change in the deflection of the membrane is determined using a laser.

Stimulating Living Material:

In order to stimulate the cell layer(s), thrombin is added to the medium to give final concentrations of 0-100 U/ml. 20 U/ml of culture medium are added first of all. The thrombin binds to the thrombin receptors and induces cell contraction. The membrane rises further to a slight extent. In this connection, the cells contract with a time constant which is typical. After a theoretically infinite measuring time, the measured change in the deflection of the membrane reaches a typical saturation value. Further quantities of thrombin are added consecutively to the cell culture medium above the membrane in increasing order, i.e. initially 50 U/ml and then 100 U/ml. The changes in the deflection of the membrane are measured correspondingly.

In each case, the cells are permitted to develop the contraction until, in the simplest case by way of fitting an exponential function, there is no need to await the achievement of the final value but, instead, the final measurement of the achievable concentration can be approximated. This decreases the measuring time. Both the characteristic onset time for the thrombin-mediated contraction effect and the maximum contraction which can be achieved in association with this contraction can be calculated from the exponential function which has been fitted.

Consecutively Detaching the Living Material and ECM from the Membrane (Calibration):

The cells are detached from the ECM by adding EDTA (0.1% by weight) for a reaction time of 2 min. The same volumes as the quantities of liquid which are added in this connection are simultaneously removed once again at another site in the measuring chamber. The cells become detached with a typical time constant. As a consequence of this, the tension in the membrane changes since the forces acting on the membrane as a result of the cell contraction are no longer present; this means that the membrane becomes further deflected once again. In this case, too, an exponential function is approximated and the time constant and the final value of the tension recovery can be calculated approximately.

The ECM which is adhering to the membrane still maintains a part of the tension which arose during the cell growth. Adding trypsin (0.2% by weight) for a reaction time of 15 min. degrades the ECM. In this case, too, the same quantity of liquid is simultaneously removed once again. The ECM proteins which can be degraded by trypsin are destroyed. The membrane is further deflected. The change in the deflection of the membrane is measured.

The residue of the ECM which still remains on the membrane, and which was not degraded by trypsin, is subsequently broken down with collagenase. This can be followed by further degradation processes so as to ensure that no matrix any longer remains on the membrane. After this (these) degradation(s), the membrane is in approximately the same tension state as it had before the cells had grown to confluence on the membrane and the matrix had formed.

In order to determine the initial tension of the membrane (calibration), the hydrostatic pressure above the membrane is now increased by adding DPBS. The added quantity of DPBS is about 8 μl resulting in the middle of the membrane being further deflected by about 40 μm. The initial tension in the membrane is determined from this so-called “calibration pressure jump” and the change in deflection which is measured. Since the respective deflection of the membrane was measured at each preceding step from the time the cells were introduced, is now possible to calculate back directly to the tension which was produced by the cells, to the changes in the tension of the cells, and their time constant, which were produced by thrombin, to the change in the tension of the ECM (ECM degradation), and its time constants, which was produced by trypsin, etc.

The given deflection state of the membrane is measured using a laser beam which is directed from below, i.e. outside the device, in a planar manner toward the middle of the membrane and reflected from this point. The reflected beam is registered by a four-quadrant diode. Displacement of the distribution of intensity between the quadrants makes it possible to precisely calculate the deflection of the membrane. The evaporation of small quantities of aqueous liquid above the membrane, which evaporation occurs during the measurements, some of which can last for a few hours, and which would become evident as a change in the deflection of the membrane, is offset by externally adding water to the medium. At the same time, the osmotic pressure of the measured solution remains constant during the measurement.

Legends to Figures:

FIG. 1: Diagram of a device for measuring forces of living material, containing a mounting (1), a membrane (2) with living material (4) applied to it, and a sensor (3). The broken line represents the fill height of the liquid column (5) above the membrane.

FIG. 2: Diagram of the procedure for training living material by means of external physical stimuli. Immersing a plunger (6) into the liquid column (5) above the membrane (2), which is attached to a mounting (1) and on which living material (4) is applied, increases the pressure in the device. Lowering (raising) the plunger by Δh leads to a change in the deflection of the membrane by Δb, which, for its part, leads to a change in the tensile stress or compressive stress in the living material. The plunger can be raised and lowered during the culturing of the living material on the membrane and is depicted as a function of the time (t), by way of example in the form of a sinusoidal curve (7).

FIG. 3: A) Diagram of the living material containing cells (1), adhesion molecules (2) and extracellular matrix (3) on a membrane (4). B) Diagram of the changes in the living material resulting from the addition of external chemical stimulants.

FIG. 4: Representation of a dose-effect curve of thrombin, ETDA and trypsin as a function of the intrinsic forces of living material (MPa) which were measured over time(s).

FIG. 5: Histogram depicting the change in the intrinsic forces (MPa) of living material in the presence of appropriate quantities (U/ml) of thrombin, EDTA and trypsin.

FIG. 6: Histogram depicting the relaxation times (min) of living material in dependence on the addition of appropriate quantities (U/ml) of thrombin, EDTA or trypsin.

FIG. 7: Representation of the intrinsic peristalsis (intrinsic contraction and relaxation) of bovine aorta endothelial cells (BAEC) as a function of the intrinsic forces of living material (MPa) which were measured over time(s), with a period length of 55 s.

1. A device for measuring forces of living material containing a mounting, an elastically deformable membrane and a sensor for measuring the change of forces acting on the membrane, comprising a membrane which is arranged on a mounting, which is stretched in a planar manner, which is freely accessible from both sides and which can be deformed elastically by applying a physical force, and which can be bonded to the living material after the latter has been applied to this membrane.

2. The device as claimed in claim 1, characterized in that the membrane exhibits a hydrophilic surface and/or a surface which is suitable for adhering and/or culturing living material, and/or is correspondingly treated and/or supplied with an adhesion-mediating substance.

3. The device as claimed in either claim 1 or 2, characterized in that the membrane has any arbitrary shape, being preferably circular, hemispherical, spherical, rectangular and/or square.

4. The device as claimed in one of claims 1 to 3, characterized in that the membrane is pore-free and/or tear-resistant and/or biologically inert.

5. The device as claimed in one of claims 1 to 4, characterized in that the thickness of the membrane is in the range from 0.1 to 10 μm, preferably of from 0.5 to 5 μm, particularly preferably 1 μm, with the ratio of the thickness of the membrane to the diameter or circumference or edge length of the membrane having a value in the range from 6×10⁻⁶ to 6×10⁻⁴, preferably of from 3×10⁻⁵ to 3×10⁻⁴, and particularly preferably 6×10⁻⁵.

6. The device as claimed in one of claims 1 to 5, characterized in that the value of the modulus of elasticity of the membrane at 25° C. is in the range from about 1000 to 10 000 MPa, preferably of from about 2500 to 6500 MPa, and particularly preferably of about 3900 MPa.

7. The device as claimed in one of claims 1 to 6, characterized in that the membrane contains enzymically degradable material which is introduced and/or applied into and/or onto the membrane.

8. The device as claimed in one of claims 1 to 7, characterized in that collagen, elastin and/or fibrinogen and/or combinations thereof is/are introduced and/or applied into and/or onto the membrane.

9. A method for measuring forces of living material, characterized in that laterally acting intrinsic forces of the living material are transmitted directly, where appropriate before, during and/or after stimulation of the material with external stimuli, to an elastically deformable membrane and the resulting change in the deflection of the membrane is registered quantitatively.

10. The method as claimed in claim 9, characterized in that
a) an elastically deformable membrane is stretched in a planar manner on a mounting such that it is freely accessible from both sides,
b) the membrane is elastically deformed by applying a physical force,
c) living material is applied to the elastically deformed membrane and a bond is formed between the living material and the membrane by means of culturing the living material and/or an adhesion mediation in and/or on the membrane,
d) where appropriate, the living material is subjected, during and/or after the formation of the bond with the membrane, to external forces which are applied constantly and/or in a pulsating manner and/or in an oscillating manner,
e) the lateral intrinsic forces which emanate from the living material are transmitted to the membrane,
f) where appropriate, additional external stimuli are exerted on the living material, and
g) the forces and/or force changes of the living material which have been transmitted to the membrane, and the time constants and/or relaxation times which are associated therewith, are quantitatively determined continuously, as changes in the deflection of the membrane, using a sensor.

11. The method as claimed in either claim 9 or 10, characterized in that the living material which is applied to the membrane contains whole cells, one or more cell layer(s), secreted cell material, preferably extracellular matrix, cell constituents and/or matrix constituents.

12. The method as claimed in one of claims 9 to 11, characterized in that the whole cells are fibroblasts and/or muscle cells and/or endothelial cells.

13. The method as claimed in one of claims 9 to 12, characterized in that genetically altered living material is employed.

14. The method as claimed in one of claims 9 to 13, characterized in that antibodies which bind specifically to intercellular and/or cell matrix adhesion proteins and/or cytoskeletal proteins are employed.

15. The method as claimed in one of claims 9 to 14, characterized in that the membrane is adhered to the mounting while it is being and/or after it has been stretched in a planar manner.

16. The method as claimed in one of claims 9 to 15, characterized in that the membrane is elastically deformed by being overlayed with a liquid column.

17. The method as claimed in one of claims 9 to 16, characterized in that a hydrostatic pressure, corresponding to a liquid column having a fill height in the range from about 0.1 to 50 mm, preferably of from 0.5 to 10 mm, and particularly preferably of 2 mm, is applied to the membrane, at a modulus of elasticity of about 3900 MPa.

18. The method as claimed in one of claims 9 to 17, characterized in that the liquid column is a medium which is suitable for culturing the living material.

19. The method as claimed in one of claims 9 to 18, characterized in that the living material is subjected to external mechanical, electrical and/or magnetic forces.

20. The method as claimed in one of claims 9 to 19, characterized in that the living material is subjected to external hydrostatic pressure changes by a plunger being immersed, cyclically and with changeable amplitude and frequency, into the liquid column over the membrane, and/or a negative pressure being applied to the membrane.

21. The method as claimed in one of claims 9 to 20, characterized in that the living material is subjected to an external addition of chemical and/or biochemical and/or biological compounds, preferably in the form of an aqueous solution.

22. The method as claimed in one of claims 9 to 21, characterized in that, as a result of adding aqueous solutions containing chemical and/or biochemical and/or biological compounds, the hydrostatic pressure above the membrane is kept constant by simultaneously withdrawing a corresponding quantity of aqueous solution which is present above the membrane.

23. The method as claimed in one of claims 9 to 22, characterized in that thrombin, trypsin, EDTA or collagenase and/or combinations thereof are added as chemical and/or biochemical compounds, and/or genetic material and/or specific antibodies are added as biological compounds.

24. The method as claimed in one of claims 9 to 23, characterized in that the time course of the forces which are transmitted to the membrane is measured, preferably continuously, over a period of from 1 second to several hours.

25. The method as claimed in one of claims 9 to 24, characterized in that intrinsic forces and/or force changes emanating from the living material are measured in a range from 0 to 5000 mPa·m, preferably of from 0 to 500 mPa·m.

26. The method as claimed in one of claims 9 to 25, characterized in that the forces and/or force changes are measured by sampling the membrane in a manner which does not involve any contact.

27. The use of the device as claimed in one of claims 1 to 8 for measuring lateral intrinsic forces of living material.

28. The use of the device as claimed in one of claims 1 to 8 for identifying chemical and/or biochemical and/or biological compounds which influence the lateral intrinsic forces of living material.
